(19) United States
(12) Patent Application Publication
(10) Pub. No.: US 2025/0009215 A1
(43) Pub. Date: Jan. 9, 2025

(54) IMAGE PROCESSING DEVICE, PHOTOTHERAPY SYSTEM, IMAGE PROCESSING METHOD, COMPUTER-READABLE RECORDING MEDIUM, AND PHOTOTHERAPY METHOD

(71) Applicant: OLYMPUS CORPORATION, Tokyo (JP)

(72) Inventor: Chikashi OTA, Tokyo (JP)

(73) Assignee: OLYMPUS CORPORATION, Tokyo (JP)

(21) Appl. No.: 18/897,728

(22) Filed: Sep. 26, 2024

Related U.S. Application Data
(63) Continuation of application No. PCT/JP2022/024572, filed on Jun. 20, 2022.

(51) Int. Cl. A61B 1/06; A61N 5/06

(52) U.S. Cl. CPC A61B 1/0638; A61B 1/063; A61B 1/0646; A61N 5/062

(57) ABSTRACT

An image processing device includes a processor including hardware, the processor being configured to: generate a white light image based on a white light which is made of a light having a wavelength band of visible light range; generate an absorption band light image indicating an absorption degree of a light having a wavelength band of Soret band which is absorbed by a drug accumulated in a target region for treatment; and generate a display image based on the white light image and the absorption band light image.

### CROSS-REFERENCE TO RELATED APPLICATION

This application is a continuation of International Application No. PCT/JP2022/024572, filed on Jun. 20, 2022, the entire contents of which are incorporated herein by reference.

### BACKGROUND

### 1. Technical Field

The present disclosure relates to an image processing device, a phototherapy system, an image processing method, a computer-readable recording medium, and a phototherapy method.

### 2. Description of the Related Art

In recent years, research has been going on about photoimmunotherapy (PIT) in which an antibody-drug is specifically bound to the protein substance of cancer cells and the antibody-drug is activated using exposure to a near-infrared light representing the treatment light, so that the cancer cells are destroyed (for example, refer to Japanese Patent Application Laid-open No. 2017-71654, International Laid-open Pamphlet No. 2019/215905, International Laid-open Pamphlet No. 2021/038913, and K. Sato et al, ACS Cent. Sci. 2018, 4, 1559-1569. and F. Inagaki, et al, Cancer Science, 2021; 112:1326-1330.). The antibody-drug that is irradiated with the near-infrared light absorbs the light energy, undergoes molecular oscillation, and produces heat. That heat causes destruction of the cancer cells. At that time, as a result of being subjected to excitation, the antibody-drug emits fluorescence. The intensity of the fluorescence is used as the index for the effectiveness of treatment. For example, in International Laid-open Pamphlet No. 2019/215905 and International Laid-open Pamphlet No. 2021/038913, it is disclosed that a light having the same wavelength band as the treatment light is applied to illuminate the antibody-drug, and the accumulation of the antibody-drug and the treatment progress is confirmed.

### SUMMARY

In some embodiments, an image processing device includes a processor including hardware, the processor being configured to: generate a white light image based on a white light which is made of a light having a wavelength band of visible light range; generate an absorption band light image indicating an absorption degree of a light having a wavelength band of Soret band which is absorbed by a drug accumulated in a target region for treatment; and generate a display image based on the white light image and the absorption band light image.

In some embodiments, a phototherapy system includes: a white light emitter configured to emit a white light made of a light having a wavelength band of visible light range; an absorption band light emitter configured to emit an absorption band light made of a light having a wavelength band of Soret band which is absorbed by a drug accumulated in a target region for treatment; an image processing device configured to generate an image based on either the white light or the absorption band light; and a display configured to display the image generated by the image processing device, the image processing device including a processor including hardware, the processor being configured to: generate a white light image based on the white light; generate an absorption band light image indicating an absorption degree of the absorption band light, and generate a display image based on the white light image and the absorption band light image.

In some embodiments, an image processing method includes: generating a white light image based on a white light which is made of a light having a wavelength band of visible light range; generating an absorption band light image indicating an absorption degree of a light having a wavelength band of Soret band which is absorbed by a drug accumulated in a target region for treatment; and generating a display image based on the white light image and the absorption band light image.

In some embodiments, provided is a non-transitory computer-readable recording medium with an executable program stored thereon, the program causes a computer to execute: generating a white light image based on a white light which is made of a light having a wavelength band of visible light range; generating an absorption band light image indicating an absorption degree of a light having a wavelength band of Soret band which is absorbed by a drug accumulated in a target region for treatment; and generating a display image based on the white light image and the absorption band light image.

In some embodiments, a phototherapy method includes: administering a drug for phototherapy to a target region for treatment; applying an absorption band light to the target region for treatment to obtain an absorption band light image that indicates an absorption degree of the absorption band light, the absorption band light including a wavelength band of Soret band and having a wavelength band lower than an excitation wavelength of the drug; generating a display image that includes the absorption band light image; displaying the display image; observing the absorption band light image to confirm an amount of accumulation of the drug; and applying a treatment light onto the target region for treatment to cause the drug that is bound to the target region for treatment to react.

The above and other features, advantages and technical and industrial significance of this disclosure will be better understood by reading the following detailed description of presently preferred embodiments of the disclosure, when considered in connection with the accompanying drawings.

### BRIEF DESCRIPTION OF THE DRAWINGS

FIG. 1 is a diagram illustrating an overall configuration of an endoscope system according to an embodiment of the disclosure;

FIG. 2 is a block diagram illustrating an overall configuration of the endoscope system according to the embodiment of the disclosure;

FIG. 3 is a diagram for explaining a front-end configuration of an endoscope according to the embodiment of the disclosure;

FIG. 4 is a diagram for explaining a configuration of an optical system of the endoscope;

FIG. 5 is a diagram that schematically illustrates a configuration of a pixel portion according to the embodiment;

FIG. 6 is a diagram that schematically illustrates a configuration of a color filter according to the embodiment;

FIG. 7 is a diagram that schematically illustrates the sensitivity characteristics of each filter;

FIG. 8A is a diagram that schematically illustrates the signal value of G pixels in the imaging element according to the embodiment;

FIG. 8B is a diagram that schematically illustrates the signal value of R pixels in the imaging element according to the embodiment;

FIG. 8C is a diagram that schematically illustrates the signal value of B pixels in the imaging element according to the embodiment;

FIG. 9 is a diagram for explaining an example of the wavelength bands of the lights used as the treatment light and the absorption band light;

FIG. 10 is a diagram illustrating an exemplary flow of the treatment performed using the endoscope according to the embodiment of the disclosure;

FIG. 11 is a flowchart for explaining an example of the operations performed in the endoscope system according to the embodiment of the disclosure;

FIG. 12 is a diagram for explaining an example of display images during the treatment;

FIG. 13 is a diagram for explaining an exemplary display image according to a first modification example;

FIG. 14 is a diagram for explaining an exemplary display image according to a second modification example; and

FIG. 15 is a block diagram illustrating an overall configuration of an endoscope system according to a third modification example.

### DETAILED DESCRIPTION

An illustrative embodiment (hereinafter, called an “embodiment”) of the disclosure is described below. In the embodiment, as an example of a system that includes a phototherapy device according to the disclosure, the explanation is given about a medical endoscope system in which in-vivo images of a patient are taken and are displayed. Meanwhile, the disclosure is not limited by the embodiment described below. Moreover, in the drawings, identical constituent elements are referred to by the same reference numerals.

### EMBODIMENT

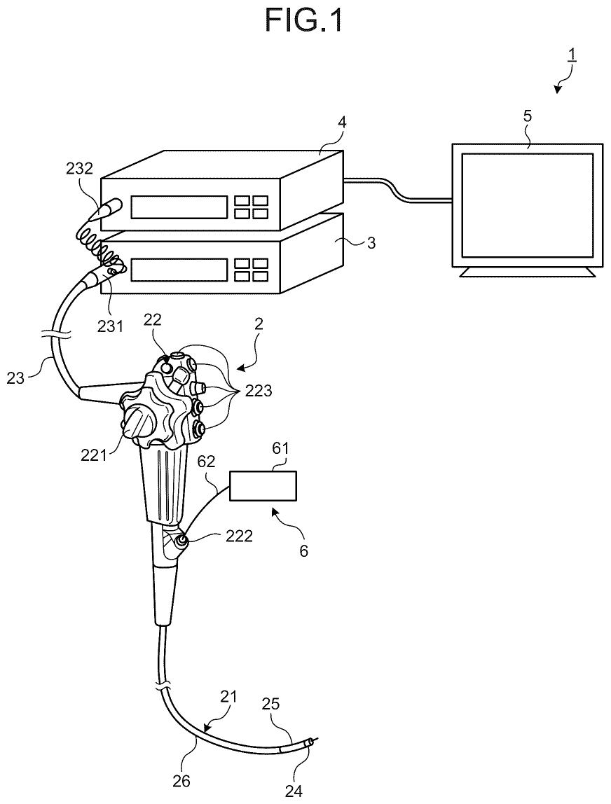

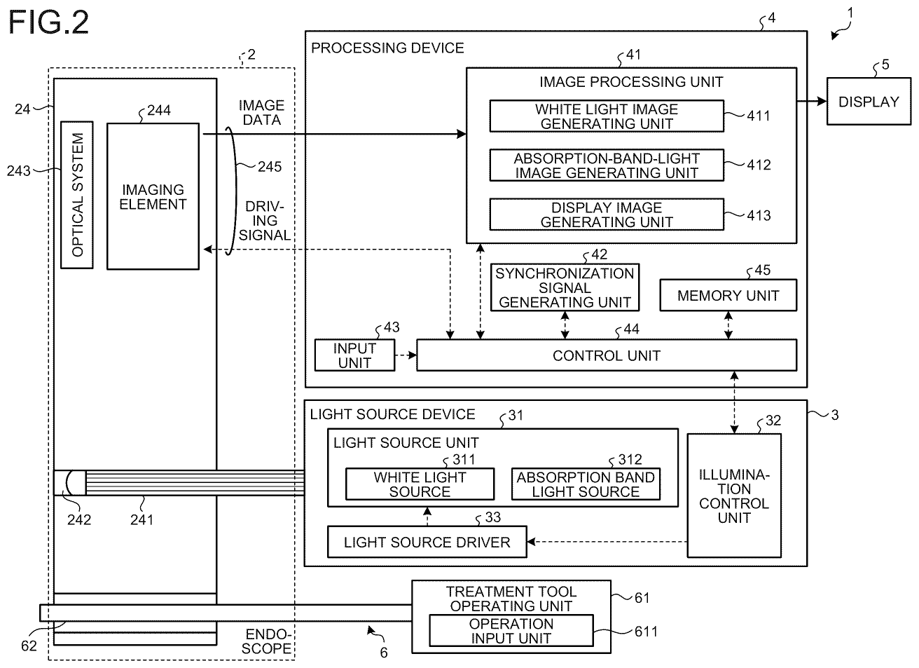

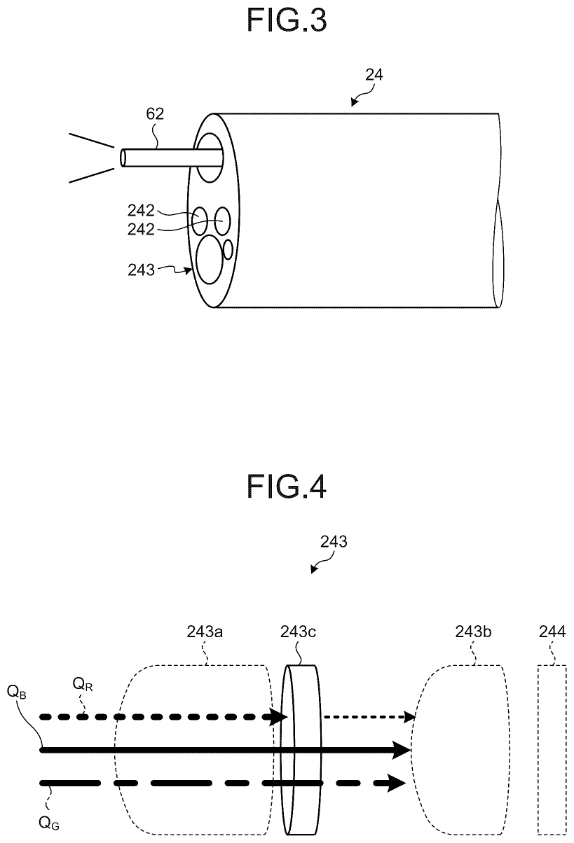

FIG. 1 is a diagram illustrating an overall configuration of an endoscope system according to the embodiment of the disclosure. FIG. 2 is a block diagram illustrating an overall configuration of the endoscope system according to the present embodiment. FIG. 3 is a diagram for explaining a front-end configuration of an endoscope according to the embodiment.

An endoscope system 1 illustrated in FIGS. 1 and 2 includes: an endoscope 2 that, when the front end portion thereof is inserted inside the subject, takes in-vivo images of the subject; a light source device 3 that generates an illumination light to be emitted from the front end of the endoscope 2; a processing device 4 that performs predetermined signal processing with respect to imaging signals that are obtained by the endoscope 2 by performing imaging, and that comprehensively controls the overall operations of the endoscope system 1; a display 5 that displays in-vivo images generated as a result of the signal processing performed by the processing device 4; and a treatment tool device 6.

The endoscope 2 includes: a flexible and elongated insertion portion 21; an operating unit 22 that is connected to the proximal end of the insertion portion 21 and that receives input of various operation signals; and a universal cord 23 that extends from the operating unit 22 in the opposite direction to the direction of extension of the insertion portion 21, and that has various built-in cables connected to the light source device 3 and the processing device 4.

The insertion portion 21 includes the following: a front end portion 24 that has a built-in imaging element 244 in which pixels meant for receiving light and generating signals according to photoelectric conversion are arranged in a two-dimensional manner; a freely-bendable curved portion 25 that is made of a plurality of bent pieces; and a flexible tube 26 that is a flexible and long tube connected to the proximal end of the curved portion 25. The insertion portion 21 is inserted into the body cavity of the subject. Then, using the imaging element 244, the insertion portion 21 takes images of the body tissue present at such positions inside the photographic subject up to which the outside light does not reach.

The operating unit 22 includes the following: a bending knob 221 that makes the curved portion 25 bend in the vertical direction and the horizontal direction; a treatment tool insertion portion 222 through which a treatment tool such as a treatment-light application device, biopsy forceps, an electrical scalpel, or an inspection probe is inserted into the body cavity of the subject; and a plurality of switches 223 representing operation input units that receive input of operation instruction signals regarding the peripheral devices including not only the processing device 4 but also an insufflation unit, a water supply unit, and a screen display control. The treatment tool inserted from the treatment tool insertion portion 222 passes through a treatment tool channel (not illustrated) in the front end portion 24 and comes out from the opening of the front end portion 24 (see FIG. 3).

The universal cord 23 at least has a built-in light guide 241 and a built-in cable assembly 245, which has one or more cables bundled therein. The universal cord 23 is branched at the end portion on the opposite side to the side of connection with the operating unit 22. At the branched end portion of the universal cord 23, a connector 231 is disposed that is detachably attachable to the light source device 3, and a connector 232 is disposed that is detachably attachable to the processing device 4. From the end portion of the connector 231, some part of the light guide 241 extends out. The universal cord 23 propagates the illumination light, which is emitted from the light source device 3, to the front end portion 24 via the connector 231 (the light guide 241), the operating unit 22, and the flexible tube 26. Moreover, the universal cord 23 transmits the image signals, which are obtained as a result of the imaging performed by the imaging element 244 that is disposed in the front end portion 24, to the processing device 4 via the connector 232. The cable assembly 245 includes a signal line for transmitting imaging signals; a signal line for transmitting driving signals meant for driving the imaging element 244; and a signal line for sending and receiving information such as the specific information related to the endoscope 2 (the imaging element 244). In the present embodiment, the explanation is given under the premise that a signal line is used for transmitting electrical signals. Alternatively, a signal line can be used for transmitting optical signals, or can be used for transmitting signals between the endoscope 2 and the processing device 4 in a wireless manner.

The front end portion 24 is made of fiberglass, and includes the following: the light guide 241 that constitutes a light guiding path for the light generated by the light source device 3; an illumination lens 242 that is disposed at the front end of the light guide 241; an optical system 243 that collects light; and the imaging element 244 that is disposed at the image formation position of the optical system 243 and that receives the light collected by the optical system 243, performs photoelectric conversion, and performs predetermined signal processing with respect to electrical signals.

The optical system 243 is configured using one or more lenses. The optical system 243 forms an observation image on the light receiving surface of the imaging element 244. Meanwhile, the optical system 243 can also be equipped with the optical zooming function for varying the angle of view and the focusing function for varying the focal point.

FIG. 4 is a diagram for explaining a configuration of the optical system of the endoscope. The optical system 243 includes two lenses 243a and 243b, and includes a cutoff filter 243c that is disposed in between the lenses 243a and 243b. The cutoff filter 243c cuts off the light having the wavelength band equal to or greater than the wavelength band of the treatment light. That is, the cutoff filter 243c allows the passage of the light having the wavelength band smaller than the wavelength band of the treatment light. For example, in the case of implementing photoimmunotherapy (PIT), the light having the wavelength band equal to or greater than 680 nm is cut, and the light having the wavelength band smaller than 680 nm passes through the cutoff filter 243c. Due to the filter characteristics thereof, the cutoff filter 243c allows the passage of some of the light having the wavelength band equal to or greater than 680 nm (see FIG. 4). For example, a light Q_{R} having the wavelength band of the red color gets cut off by the cutoff filter 243c. On the other hand, a light Q_{B} having the wavelength band of the blue color and a light Q_{G} having the wavelength band of the green color pass through the cutoff filter 243c.

It is desirable that the cutoff filter 243c is a filter having a high OD value.

Meanwhile, the configuration of the optical system illustrated in FIG. 4 is only exemplary, and it is possible to make various changes in the design.

The imaging element 244 performs photoelectric conversion with respect to the light coming from the optical system 243, and generates electrical signals (image signals). The imaging element 244 includes: a pixel portion in which a plurality of pixels, each of which includes a photodiode for accumulating the electrical charge according to the amount of light and includes a capacitor for converting the electrical charge transferred from the photodiode into a voltage level, is arranged as a two-dimensional matrix; and includes a color filter disposed in each pixel. In the imaging element 244, each pixel performs photoelectric conversion with respect to the incoming light coming via the optical system 243 and generates an electrical signal. Then, the imaging element 244 sequentially reads the electrical signals generated by arbitrarily-set target pixels for reading from among a plurality of pixels, and outputs those electrical signals as image signals. The imaging element 244 is implemented using, for example, a CCD image sensor (CCD stands for Charge Coupled Device) or a CMOS image sensor (CMOS stands for Complementary Metal Oxide Semiconductor).

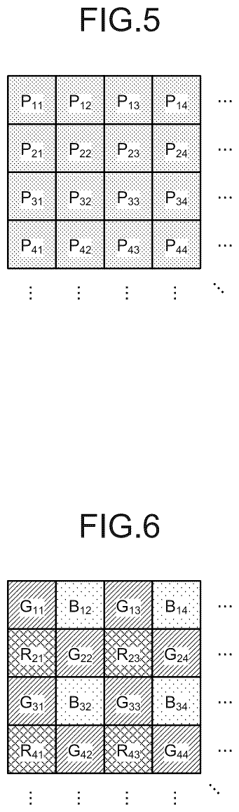

FIG. 5 is a diagram that schematically illustrates a configuration of the pixel portion of the imaging element 244. In the pixel portion, a plurality of pixels Pₙₘ (where “n” and “m” are integers equal to or greater than “1”), each including a photodiode for accumulating the electrical charge according to the amount of light, is arranged as a two-dimensional matrix. Under the control of a control unit 44, the pixel portion sequentially reads, as image data, the image signals of such pixels Pₙₘ which, from among a plurality of pixels Pₙₘ, are included in an arbitrarily-set target reading region for reading, and outputs the image signals to the processing device 4.

FIG. 6 is a diagram that schematically illustrates a configuration of the color filter of the imaging element 244. The color filter is configured according to the Bayer layout in which a 2×2 block is treated as a single unit. The color filter is configured using a filter R that allows a passage of a light having a wavelength band of red color, a filter G that allows a passage of a light having a wavelength band of green color, and a filter B that allows a passage of a light having a wavelength band of blue color. Meanwhile, with reference to FIG. 6, the reference numeral assigned to each filter (for example, G₁₁) corresponds to a pixel Pₙₘ and indicates that the concerned filter is placed at the corresponding pixel position.

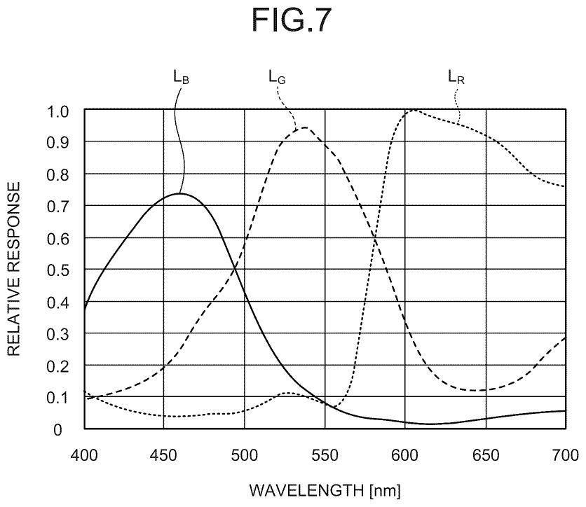

FIG. 7 is a diagram that schematically illustrates the sensitivity characteristics of each filter. In FIG. 7, the horizontal axis represents the wavelength (nm) and the vertical axis represents the transmission characteristics (the sensitivity characteristics). Moreover, in FIG. 7, a curved line L_{B} represents the transmission characteristics of the filters B, a curved line L_{G} represents the transmission characteristics of the filters G, and a curved line L_{R} represents the transmission characteristics of the filters R.

The filters B allow the passage of the light having the wavelength band of the blue color (see the curved line L_{B} in FIG. 7). The filters G allows the passage of the light having the wavelength band of the green color (see the curved line L_{G} in FIG. 7). The filter R allows the passage of the light having the wavelength band of the red color (see the curved line L_{R} in FIG. 7). In the following explanation, each pixel Pₙₘ in which the filter R is placed at the light receiving surface is referred to as an R pixel; each pixel Pₙₘ in which the filter G is placed at the light receiving surface is referred to as a G pixel; and each pixel Pₙₘ in which the filter B is placed at the light receiving surface is referred to as a B pixel.

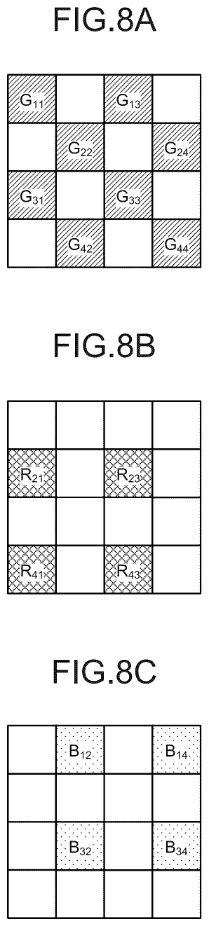

Thus, when a photographic subject image formed by the optical system 243 is received, the imaging element 244 that is configured as explained above generates color signals of the G pixels, the R pixels, and the B pixels (i.e., generates G signals, R signals, and B signals) (see FIGS. 8A to 8C).

Meanwhile, the endoscope 2 includes a memory (not illustrated) for storing data that contains an execution program and a control program written for enabling the imaging element 244 to perform various operations, and that contains identification information of the endoscope 2. The identification information contains the specific information (ID), the model year, the specifications information, and the transmission method of the endoscope 2. Moreover, the memory can also store therein, on a temporary basis, the image data generated by the imaging element 244.

Given below is the explanation of a configuration of the light source device 3. The light source device 3 includes a light source unit 31, an illumination control unit 32, and a light source driver 33. Under the control of the illumination control unit 32, the light source unit 31 sequentially switches the illumination light and emits it onto the photographic subject (subject).

The light source unit 31 is configured using a light source and one or more lenses, and emits a light (illumination light) when the light source is driven. The light generated by the light source unit 31 is emitted from the front end of the front end portion 24 toward the photographic subject via the light guide 241. The light source unit 31 includes a white light source 311 and an absorption band light source 312. Herein, the light sources, the light guide 241, and the illumination lens 242 constitute an emitter. For example, the absorption band light source 312, the light guide 241, and the illumination lens 242 constitute an absorption band light emitter.

The white light source 311 emits the light having the wavelength band of the visible light range (i.e., emits a white light). The white light source 311 is implemented using an LED light source, a laser light source, a xenon lamp, or a halogen lamp.

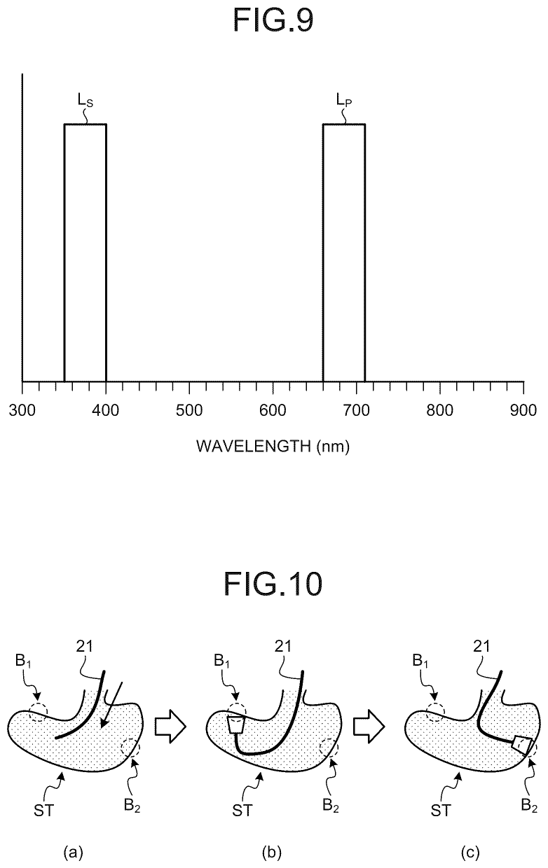

The absorption band light source 312 represents the light made of some wavelengths or some part of the wavelength band of the wavelength band of the visible light range, and emits the light of the absorption band of the antibody-drug used in photoimmunotherapy (i.e., emits an absorption band light). This absorption band represents the wavelength band of the Soret band of the antibody-drug and, for example, is equal to or smaller than 450 nm. In the antibody-drug used in photoimmunotherapy, the wavelength band of the Soret band is equal to or greater than 350 nm and equal to or smaller than 400 nm. FIG. 9 is a diagram for explaining an example of the wavelength bands of the lights used as the treatment light and the absorption band light. In FIG. 9, the vertical axis represents the relative intensity and is assumed to be equal for both lights. As the absorption band light, for example, a light L_{S} has the wavelength band equal to or greater than 350 nm and equal to or smaller than 400 nm. In the present embodiment, the explanation is given about an example in which the light L_{S} is used as the absorption band light. Meanwhile, the absorption band light source 312 is implemented using an LED light source or a laser light source.

In the case of causing excitation of the antibody-drug in photoimmunotherapy, for example, the near-infrared light having the central wavelength of 690 nm is used (for example, a light L_{P} having the wavelength band equal to or greater than 660 nm and equal to or smaller than 710 nm as illustrated in FIG. 9 is used).

In that regard, a narrow-band light having a different wavelength band than the wavelength band of the absorption band light can be used. For example, a light having the wavelength band equal to or greater than 390 nm and equal to or smaller than 445 nm can be used, or a light having the wavelength band equal to or greater than 530 nm and equal to or smaller than 550 nm can be used, or a narrow-band light formed by combining those two lights can be used. If the light having the wavelength band equal to or greater than 390 nm and equal to or smaller than 445 nm is applied and if the scattering light or the returning light is obtained, then the blood vessels in the superficial portion of the mucous membrane can be visualized with a high degree of contrast. Alternatively, if the light having the wavelength band equal to or greater than 530 nm and equal to or smaller than 550 nm is applied and if the scattering light or the returning light is obtained, then the blood vessels in the relatively deeper part of the superficial portion of the mucous membrane can be visualized with a high degree of contrast. Apart from that, if the light having the wavelength band equal to or greater than 590 nm and equal to or smaller than 620 nm is emitted, or if the light having the wavelength band equal to or greater than 620 nm and equal to or smaller than 780 nm is emitted, and if the scattering light or the returning light is obtained; then also the blood vessels in the relatively deeper part of the superficial portion of the mucous membrane can be visualized with a high degree of contrast.

Based on a control signal (modulated light signal) received from the processing device 4, the illumination control unit 32 controls the electrical energy to be supplied to the light source unit 31, controls the light source to be made to emit light, and controls the driving timing of the light source.

Under the control of the illumination control unit 32, the light source driver 33 supplies an electrical current to the light source to be made to emit light, and causes the light source unit 31 to output the light.

Given below is the explanation of a configuration of the processing device 4. The processing device 4 includes an image processing unit 41, a synchronization signal generating unit 42, an input unit 43, a control unit 44, and a memory unit 45.

The image processing unit 41 receives, from the endoscope 2, image data of the illumination light of each color as obtained by the imaging element 244 by performing imaging. If analog image data is received from the endoscope 2, then the image processing unit 41 performs A/D conversion and generates digital imaging signals. Alternatively, if image data is received in the form of optical signals from the endoscope 2, then the image processing unit performs photoelectric conversion and generates digital image data.

The image processing unit 41 performs predetermined image processing with respect to the image data received from the endoscope 2; generates an image; and outputs the image to the display 5, sets a reinforcement region determined based on the image, and calculates the time variation of the fluorescence intensity. The image processing unit 41 includes a white light image generating unit 411, an absorption-band-light image generating unit 412, and a display image generating unit 413.

The white light image generating unit 411 generates a white light image based on the image formed due to the white light. The white light image generating unit 411 generates a white light image based on the image signals of the R pixels, the image signals of the G pixels, and the image signals of the B pixels.

In the present embodiment, the absorption-band-light image generating unit 412 generates an absorption band light image that is based on an image formed due to the absorption band light and that is to be overlapped onto the white light image. The absorption-band-light image generating unit 412 uses the color signals of the B pixels and generates, for example, a grayscale absorption band light image that expresses the absorption of the absorption band light by the antibody-drug. In an absorption band light image, greater the absorption of the absorption band light at a particular spot, the smaller becomes the luminance value at that spot. The absorption-band-light image generating unit 412 generates an absorption band light image in which, smaller the luminance value, the denser becomes the shading. Hence, when an absorption band light image is a grayscale image; greater the absorption of the absorption band light, the darker becomes the image.

The display image generating unit 413 generates, as an image to be displayed in the display 5, a white light image, or an absorption band light image, or a narrow-band light image, or a superimposed image that is formed when an absorption band light image for superimposition is superimposed onto a predetermined image. Thus, a superimposed image is formed when an absorption band light image is superimposed onto an image that is based on the white light or the narrow-band light. At that time, for example, the display image generating unit 413 extracts, from the absorption band light image, a region having the luminance value equal to or greater than the luminance value equivalent to an absorption region, and superimposes the extracted region onto a white light image.

Herein, the optical system 243, the imaging element 244, and an image generating unit constitute an image obtaining unit. For example, in the case of obtaining an image formed due to the illumination of the absorption band light; the optical system 243, the imaging element 244, and the absorption-band-light image generating unit 412 constitute an absorption-band-light image obtaining unit.

The white light image generating unit 411, the absorption-band-light image generating unit 412, and the display image generating unit 413 generate images by performing predetermined image processing. Herein, the predetermined image processing indicates synchronization, gray level correction, and color correction. The synchronization represents the operation of achieving synchronization among the image data of the RGB color components. The gray level correction represents the operation of correcting the gray level of the image data. The color correction represents the operation of performing color compensation with respect to the image data. Meanwhile, the white light image generating unit 411, the absorption-band-light image generating unit 412, and the display image generating unit 413 can also perform gain adjustment according to the brightness of an image.

The image processing unit 41 is configured either using a general-purpose processor such as a central processing unit (CPU) or using a dedicated processor such as one of various arithmetic circuits, such as an application specific integrated circuit (ASIC), that implements specific functions. Moreover, the image processing unit 41 can be configured to include a frame memory for holding R image data, G image data, and B image data.

The synchronization signal generating unit 42 generates clock signals (synchronization signals) serving as the basis for the operations performed by the processing device 4, and outputs the generated synchronization signals to the light source device 3, the image processing unit 41, the control unit 44, and the endoscope 2. Herein, the synchronization signals generated by the synchronization signal generating unit 42 include a horizontal synchronization signal and a vertical synchronization signal.

Thus, the light source device 3, the image processing unit 41, the control unit 44, and the endoscope 2 perform operations in synchronization with each other based on the generated synchronization signals.

The input unit 43 is configured using a keyboard, a mouse, switches, or a touch-sensitive panel, and receives input of various signals such as an operation instruction signal that is meant for instructing the operations of the endoscope system 1. Meanwhile, the input unit 43 can also represent switches installed in the operating unit 22, or can be a portable terminal such as an external tablet computer.

The control unit 44 performs driving control of the constituent elements including the imaging element 244 and the light source device 3, and performs input-output control of information with respect to the constituent elements. The control unit 44 refers to control information data (for example, the reading timing) that is stored in the memory unit 45 and that is to be used in performing imaging control, and sends the control information data as driving signals to the imaging element 244 via predetermined signal lines included in the cable assembly 245. Moreover, the control unit 44 switches between a normal observation mode (white light observation mode), which enables observation of the images obtained in white light illumination, and an absorption band light observation mode, which enables observation of the images obtained in absorption band light illumination. The control unit 44 is configured using a general-purpose processor such as a CPU or using a dedicated processor such as one of various arithmetic circuits, such as an ASIC, that implements specific functions.

The memory unit 45 stores therein various computer programs written for causing the endoscope system 1 to perform operations, and stores therein data containing various parameters required in the operations performed by the endoscope system 1. Moreover, the memory unit 45 stores therein the identification information of the processing device 4, which contains the specific information (ID), the model year, and the specifications information of the processing device 4.

Moreover, the memory unit 45 stores therein various computer programs including an image obtaining program that is written for enabling the processing device 4 to implement an image obtaining method. The computer programs can be recorded for circulation in a computer-readable recording medium such as a hard disk, a flash memory, a CD-ROM, a DVD-ROM, or a flexible disk. Alternatively, the computer programs can be downloaded via a communication network, which is implemented using, for example, an existing public line, or a local area network (LAN), or a wide area network (WAN); and which can be a wired network or a wireless network.

The memory unit 45 is implemented using a read only memory (ROM) in which various computer programs are installed in advance, and using a random access memory (RAM) or a hard disk in which various operation parameters and data are stored.

The display 5 displays a display image corresponding to the image signal received from the processing device 4 (the image processing unit 41) via a video cable. The display 5 is configured using a monitor such as a liquid crystal display or an organic electroluminescence (EL) display.

The treatment tool device 6 includes a treatment tool operating unit 61, and includes a flexible treatment tool 62 that extends from the treatment tool operating unit 61. The treatment tool 62 represents a treatment light emitter that, when used in photoimmunotherapy, emits a light for enabling treatment (hereinafter, called the treatment light). The treatment tool operating unit 61 controls the emission of the treatment light from the treatment tool 62. The treatment tool operating unit 61 includes an operation input unit 611 that is configured using, for example, switches. In response to an input (for example, in response to the pressing of a switch) with respect to the operation input unit 611, the treatment tool operating unit 61 causes the treatment tool 62 to emit the treatment light. Meanwhile, in the treatment tool device 6, the light source that emits the treatment light either can be installed in the treatment tool 62 or can be installed in the treatment tool operating unit 61. The light source is implemented using a semiconductor laser or a light emitting diode (LED). For example, in the case of implementing photoimmunotherapy, the treatment light has the wavelength band equal to or greater than 680 nm and, for example, has the central wavelength of 690 nm (for example, the light L_{P} illustrated in FIG. 9).

Herein, the illumination optical system included in the treatment tool 62 can be configured to enable making changes in the application range of the treatment light. For example, under the control of the treatment tool operating unit 61, the illumination optical system can be configured either using an optical system in which the focal distance can be varied or using a digital micromirror device (DMD); so that it becomes possible to vary the spot diameter of the light applied onto the subject and to vary the shape of the application range.

Explained below with reference to FIG. 10 is the flow of the treatment performed using the endoscope 2. FIG. 10 is a diagram illustrating an exemplary flow of the treatment performed using the endoscope according to the embodiment of the disclosure. In FIG. 10 is illustrated an example of implementing photoimmunotherapy; and the insertion portion 21 is inserted into a stomach ST for carrying out the treatment.

Firstly, the operator inserts the insertion portion 21 into the stomach ST (see (a) in FIG. 10). At that time, the operator causes the light source device 3 to emit the white light and, while observing the white-light image that captures the inside of the stomach ST and that is displayed in the display 5, searches for the treatment position. Herein, it is assumed that the treatment is carried out for tumors B₁ and B₂ representing the target regions for treatment. At that time, an antibody-drug is administered to the tumors B₁ and B₂ representing the target regions for treatment. The antibody-drug either can be administered using the endoscope 2 or can be administered using some other device, or the patient can be asked to take in the drug.

The operator observes the white-light image and decides that the regions including the tumors B₁ and B₂ represent application regions. Moreover, as may be necessary, a narrow-band light can be applied onto the application regions, and narrow-band light images can be obtained. By checking the narrow-band light images, the operator can confirm the blood vessels in the superficial portion of the body tissue.

The operator orients the front end portion 24 toward the tumor B₁, projects the treatment tool 62 from the front end of the endoscope 2, and applies the treatment light onto the tumor B₁ (see (b) in FIG. 10). As a result of the application of the treatment light, the antibody drug that is bound to the tumor B₁ undergoes reaction, and the treatment for the tumor B₁ is carried out.

Subsequently, the operator orients the front end portion 24 toward the tumor B₂, projects the treatment tool 62 from the front end of the endoscope 2, and applies the treatment light onto the tumor B₂ (see (c) in FIG. 10). As a result of the application of the treatment light, the antibody drug that is bound to the tumor B₂ undergoes reaction, and the treatment for the tumor B₂ is carried out.

Herein, as may be necessary, the operator again applies the treatment light and confirms the effect of treatment in a repeated manner.

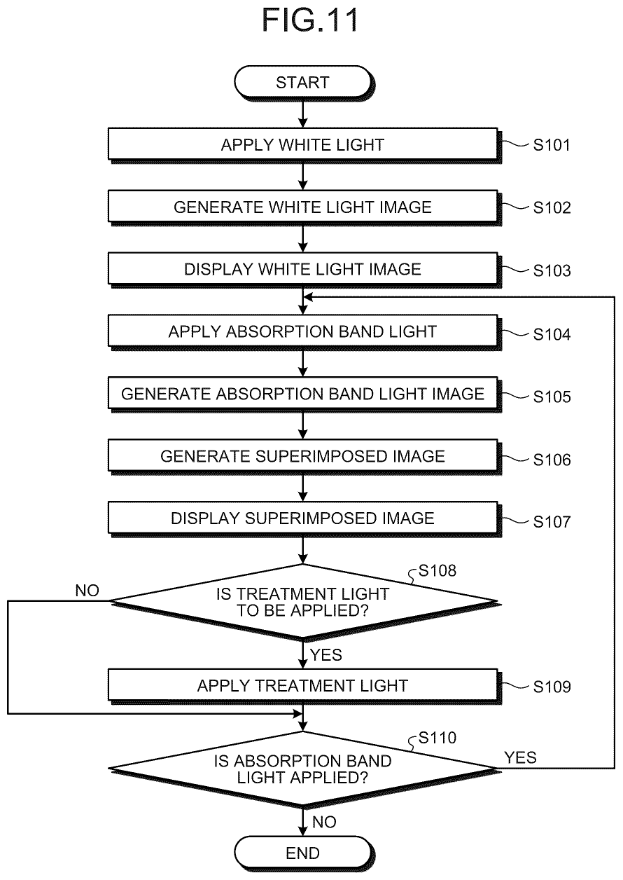

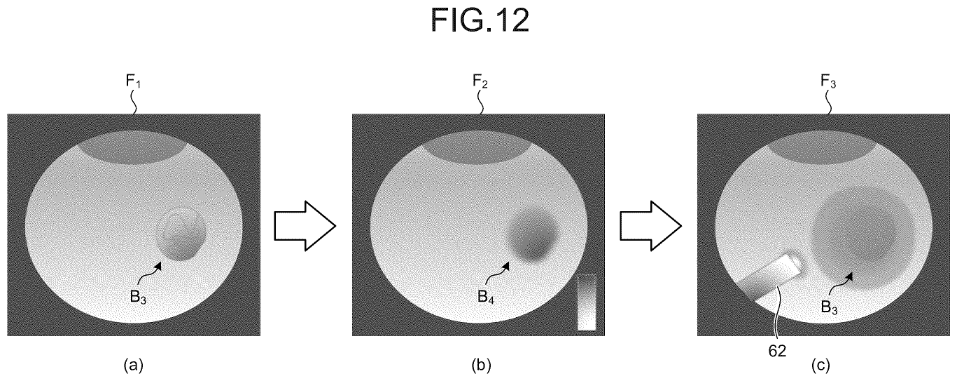

At that time, the operator applies the absorption band light onto both tumors, obtains absorption band light images, and observes the amount of the antibody-drug present in the tumors. For example, the operator applies the absorption band light before the application of the treatment light and after the treatment, and confirms the amount of accumulation of the antibody-drug at each point of time. FIG. 11 is a flowchart for explaining an example of the operations performed in the endoscope system according to the embodiment.

Firstly, due to the operation performed by the operator, the insertion portion 21 is inserted inside the subject and the white light is applied onto the body tissue inside the subject (Step S101). In the processing device 4, the white light image generating unit 411 generates a white light image based on the white light (Step S102). Then, the display image generating unit 413 generates a white light image for display, and displays it in the display 5 (Step S103). At that time, the control unit 44 sets the observation mode to the normal observation mode and displays the white light image in the display 5. For example, the control unit 44 sets the observation mode in response to a white light emission instruction.

The operator observes the displayed white light image and searches for tumors. Then, the operator administers the antibody-drug at the target regions for treatment that are found.

Subsequently, according to an operation performed by the user, the control unit 44 emits the absorption band light in such a way that the absorption band light is applied onto the treatment positions from the front end portion 24 (Step S104). Then, the absorption-band-light image generating unit 412 generates an absorption band light image based on the absorption band light (Step S105). The absorption-band-light image generating unit 412 uses the signals of the B pixels and generates an absorption band light image that is displayed to be darker when the luminance value is smaller (i.e., when the absorption degree is higher) and that is to be superimposed onto the white light image.

At that time, the control unit 44 sets the observation mode to the absorption band light observation mode. For example, the control unit 44 switches the observation mode in response to an absorption band light emission instruction.

Then, the display image generating unit 413 generates a superimposed image for display in which the absorption band light image is superimposed onto the white light image (Step S106). The control unit 44 displays the superimposed image in the display 5 (Step S107).

Meanwhile, in the case of using a narrow-band light, the display image generating unit 413 can generate a display image in which the superimposed image is further superimposed onto a narrow-band light image. In the case of using a narrow-band light image, since a blood vessel contrast image serves as the background of the image due to the narrow-band light, it becomes possible to obtain an image that enables visual confirmation of the boundary region of the antibody-drug accumulated in a cancer tissue region.

After the superimposed image is displayed, the control unit 44 determines whether or not the treatment light is to be applied according to an operation performed by the operator (Step S108). For example, the control unit 44 determines whether or not the operation input unit 611 has received an operation input, and accordingly determines whether or not the treatment light is to be applied. If it is determined that the treatment light is not to be applied (No at Step S108), then the system control proceeds to Step S110. On the other hand, if it is determined that the treatment light is to be applied (Yes at Step S108), then the system control proceeds to Step S109.

At Step S109, the treatment tool 62 is inserted inside the endoscope 2 according to an operation performed by the operator and the treatment light is applied from the treatment tool 62 onto the antibody-drug, which is bound to the cancer cells, thereby causing a reaction of the drug (a drug reaction process). In the drug reaction process, the antibody-drug is activated using exposure to a near-infrared light representing the treatment light, and the cancer cells are destroyed.

At Step S110, the control unit 44 determines whether or not the absorption band light is applied according to an operation of the operator. Thus, the control unit 44 determines whether or not the input unit 43 has received an operation input and accordingly determines whether or not the absorption band light is applied. If it is determined that the absorption band light is not applied (No at Step S110), then it marks the end of the operations. On the other hand, if it is determined that the absorption band light is applied (Yes at Step S110), then the system control returns to Step S104 and the operations are repeated. If the absorption band light is applied after the application of the treatment light; then, in the absorption band light image that is obtained, the grayscale expresses the amount of the antibody-drug reduced due to the application of the treatment light.

Explained below with reference to FIG. 12 are the display images that are displayed during absorption band light observation. FIG. 12 is a diagram for explaining an example of the display images during the treatment. Firstly, due to the application of the white light, a white light image F₁ is displayed (see (a) in FIG. 12). In the white light image F₁, a tumor B₃ is displayed. Thus, the operator observes the white light image F₁ and understands about the tumor B₃.

Then, due to the application of the absorption band light, a superimposed image F₂ is displayed in which a superimposed image indicating the absorption of the absorption band light is superimposed onto the white light image (see (b) in FIG. 12). In the superimposed image F₂, a Soret band region B₄ is displayed that corresponds to the region absorbed by the antibody-drug. The Soret band region B₄ is extracted using a superimposed image that is generated according to the luminance value of an image based on the absorption band light and that has a preset hue and grayscale assigned thereto.

Then, as a result of displaying the white light image F₃, the condition indicating that the treatment light is applied onto the tumor B₃ from the treatment tool 62 is displayed (see (c) in FIG. 12). Thus, in the white light image F₃, the tumor B₃ and the treatment tool 62 are displayed. The operator observes the white light image F₃, and applies the treatment light while observing the tumor B₃ and the treatment tool 62.

As a result of displaying the images in the display 5, the operator is allowed to understand about the amount of the antibody-drug and the effect of treatment. More particularly, the operator confirms the amount of the antibody-drug based on the grayscale of the superimposed image and determines whether or not to additionally apply the treatment light and determines the portion in which the treatment light is to be applied.

As explained above, according to the embodiment, by observing a superimposed image that is obtained by applying the absorption band light corresponding to the Soret band, it becomes possible for the operator to confirm the amount of the antibody-drug accumulated in the target region for treatment. Moreover, since the absorption band light has a different wavelength band than the wavelength band of the treatment light, it becomes possible to hold down the advancement of the treatment while the confirmation is underway. According to the present embodiment, it becomes possible to confirm the course of treatment including the accumulated amount of the antibody-drug, while holding down the advancement of the reaction during photoimmunotherapy. Furthermore, according to the present embodiment, since the advancement of the treatment is held down while the confirmation is underway, the observation period for observing the antibody-drug can be secured over a longer period of time as compared to the case in which the antibody-drug is in the excited state and the confirmation is done based on the fluorescence.

Moreover, in the embodiment described above, as a result of using the imaging element 244 that includes color filters; color signals (B pixels) indicating the absorption of the absorption band light can be obtained and an absorption band light image (a superimposed image) for expressing the absorption of the antibody-drug can be generated without having to add any new configuration.

### First Modification Example

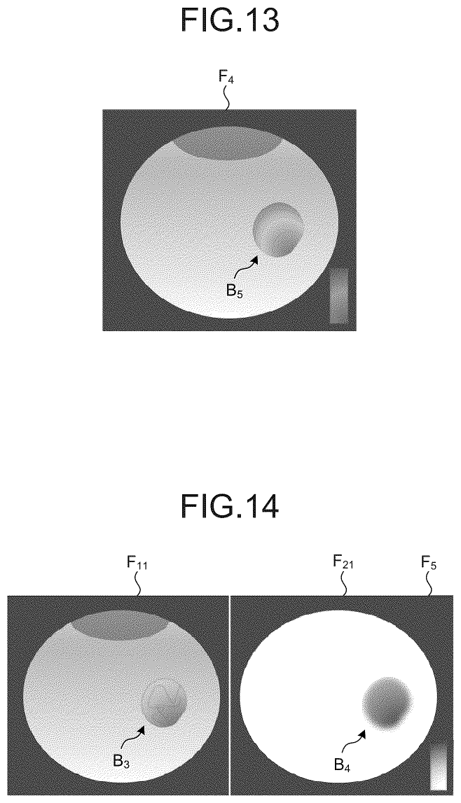

Explained below with reference to FIG. 13 is a first modification example of the embodiment. FIG. 13 is a diagram for explaining an exemplary display image according to the first modification example. An endoscope system according to the first modification example is identical to the endoscope system 1 according to the embodiment. Hence, that explanation is not given again. In the embodiment, the explanation of the absorption band light image is given about an example in which the absorption degree of the absorption band light is expressed using the grayscale of a single hue. In contrast, in the first modification example, the absorption degree is expressed using a plurality of hues.

In the first modification example, the absorption-band-light image generating unit 412 uses the signals of the B pixels and generates an absorption band light image for superimposition that is displayed by varying the hue according to the luminance value (the absorption degree). In an absorption band light image F₄ illustrated in FIG. 13, a Soret band region B₅ is displayed that corresponds to the region absorbed by the antibody-drug. The Soret band region B₅ is assigned with the hue corresponding to the luminance value of the image that is based on the absorption band light.

The display image generating unit 413 superimposes the absorption band light image onto the white light image and generates a superimposed image for display. Then, under the control of the control unit 44, the superimposed image gets displayed in the display 5.

In the first modification example explained above, in an identical manner to the embodiment, as a result of observing a superimposed image obtained by applying the absorption band light corresponding to the Soret band, it becomes possible for the operator to confirm the amount of the antibody-drug accumulated in the target region for treatment. Moreover, since the absorption band light has a different wavelength band than the wavelength band of the treatment light, it becomes possible to hold down the advancement of the treatment while the confirmation is underway. According to the first modification example, it becomes possible to confirm the course of treatment including the accumulated amount of the antibody-drug, while holding down the advancement of the reaction during photoimmunotherapy. Furthermore, the observation period for observing the antibody-drug can be secured over a longer period of time.

### Second Modification Example

Explained below with reference to FIG. 14 is a second modification example of the embodiment. FIG. 14 is a diagram for explaining an exemplary display image according to the second modification example. An endoscope system according to the second modification example is identical to the endoscope system 1 according to the embodiment. Hence, that explanation is not given again. In the embodiment, the explanation is given about an example in which an absorption band light image is superimposed onto a white light image. In contrast, in the second modification example, a display image includes a white light image and an absorption band image placed side-by-side.

In the second modification example, the display image generating unit 413 generates a display image F₅ in which a white light image Fu capturing the tumor B₃ is placed side-by-side with an absorption band light image F₂₁ capturing the Soret band region B₄.

In the second modification example explained above, the white light image is displayed separately from the absorption band light image that is obtained by applying the absorption band light corresponding to the Soret band. As a result, in an identical manner to the embodiment, it becomes possible for the operator to confirm the amount of the antibody-drug accumulated in the target region for treatment. Moreover, since the absorption band light has a different wavelength band than the wavelength band of the treatment light, it becomes possible to hold down the advancement of the treatment while the confirmation is underway. According to the second modification example, it becomes possible to confirm the course of treatment including the accumulated amount of the antibody-drug, while holding down the advancement of the reaction during photoimmunotherapy. Furthermore, the observation period for observing the antibody-drug can be secured over a longer period of time.

### Third Modification Example

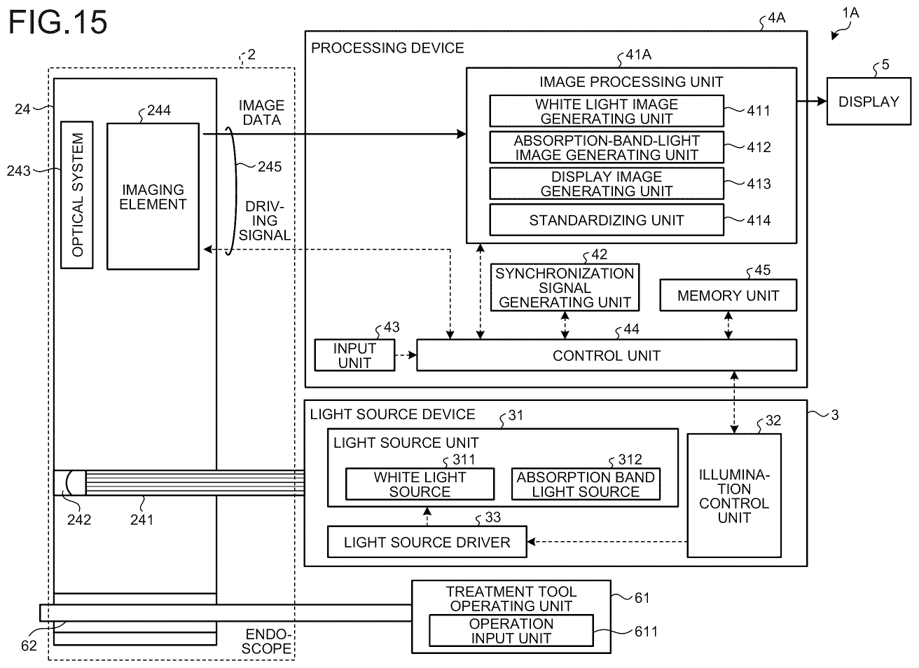

Explained below with reference to FIG. 15 is a third modification example of the embodiment. FIG. 15 is a block diagram illustrating an overall configuration of an endoscope system according to the third modification example. An endoscope system 1A according to the third modification example includes a processing device 4A in place of the processing device 4 of the endoscope system 1 according to the embodiment. Other than that, the configuration is identical and hence is not explained again. In the embodiment, the explanation is given about an example of using an image formed by superimposing an absorption band light image onto a white light image. In contrast, in the second modification example, a display image includes a white light image and an absorption band image placed side-by-side.

The processing device 4A includes an image processing unit 41A, the synchronization signal generating unit 42, the input unit 43, the control unit 44, and the memory unit 45. The synchronization signal generating unit 42, the input unit 43, the control unit 44, and the memory unit 45 are identical to the first embodiment. Hence, their explanation is not given again.

In an identical manner to the image processing unit 41, the image processing unit 41A receives, from the endoscope 2, image data of the illumination light of each color as obtained by the imaging element 244 by performing imaging; performs predetermined image processing with respect to the image data and generates an image; and outputs the image to the display 5, sets an reinforcement region determined based on the image, and calculates the time variation of the fluorescence intensity. The image processing unit 41A includes the white light image generating unit 411, the absorption-band-light image generating unit 412, the display image generating unit 413, and a standardizing unit 414.

In the third modification example, the color signals of the B pixels correspond to the antibody-drug absorption region and are treated as the signals including the region in which the reflectance undergoes a decline due to absorption. The color signals of the G pixels represent the scattering light coming from the body tissue during photoimmunotherapy, and are treated as the background that is not affected due to the absorption by the antibody-drug and as reference signals for standardizing the color signals.

The standardizing unit 414 standardizes, for example, the signals of the B pixels using the signals of the G pixels. Because of the standardization operation, the post-standardization signal value (i.e., the standardized value) of the B pixels becomes smaller in the area in which the absorption band light is absorbed.

The absorption-band-light image generating unit 412 generates an absorption band light image in which, smaller the standardized value, the darker becomes the color. At that time, a background image can be generated based on the signals of the G pixels.

The display image generating unit 413 superimposes the absorption band light image, which is generated by the absorption-band-light image generating unit 412, onto the white light image and generates a superimposed image.

In the third embodiment, an image is generated in which, smaller the standardized value, the darker is the grayscale of the contrast. For that reason, greater the absorption of the absorption band light, the darker becomes the color of the image. Meanwhile, it is also possible to use the absorption band light image according to the first modification example or to use the display image according to the second modification example.

In the third modification example explained above, the white light image is displayed separately from the absorption band light image that is obtained by applying the absorption band light corresponding to the Soret band. As a result, in an identical manner to the embodiment, it becomes possible for the operator to confirm the amount of the antibody-drug accumulated in the target region for treatment. Moreover, since the absorption band light has a different wavelength band than the wavelength band of the treatment light, it becomes possible to hold down the advancement of the treatment while the confirmation is underway. According to the third modification example, it becomes possible to confirm the course of treatment including the accumulated amount of the antibody-drug, while holding down the advancement of the reaction during photoimmunotherapy. Furthermore, the observation period for observing the antibody-drug can be secured over a longer period of time.

Moreover, according to the third modification example, as a result of standardizing the signal value of the B pixels, it becomes possible to generate a superimposed image in which the variability in the illumination intensity is held down and the contribution of light absorption is highlighted.

Furthermore, according to the third modification example, as a result of using the imaging element 244 that includes color filters; color signals (B pixels) indicating the absorption of the absorption band light can be obtained, color signals (G pixels) used for the background and for reference can be obtained, and an absorption band light image (a superimposed image) for expressing the absorption of the antibody-drug can be generated without having to add any new configuration.

In the third modification example, the explanation is given about an example in which the standardizing unit 414 performs standardization using the signals of the G pixels. However, that is not the only possible case. Alternatively, the signals of the B pixels can be standardized by performing division using the sum of the signals of the R pixels, the G pixels, and the B pixels, or the signals of the B pixels can be standardized by performing division using the sum of the signals of the R pixels and the G pixels.

In the embodiment described above, the explanation is given about an example in which the light source device 3 is a separate device from the processing device 4. Alternatively, the light source device 3 and the processing device 4 can be integrated into a single device. Moreover, in the embodiment, the explanation is given about an example in which the treatment light is applied using a treatment tool. Alternatively, the light source device 3 can be configured to emit the treatment light.

Furthermore, in the embodiment described above, the imaging element 244 can be configured using a multi-band image sensor, so that the lights having a plurality of mutually different wavelength bands can be individually obtained.

Moreover, in the embodiment described above, in order to confirm the presence of the antibody-drug, the configuration can allow emission of the excitation light for causing excitation of the antibody-drug. At that time, the excitation light and the treatment light either can have the same wavelength band (the same central wavelength) or can have mutually different wavelength bands (mutually different central wavelengths). Meanwhile, when the excitation light is to be used as the treatment light too, the treatment light (the excitation light) can be applied either using the treatment tool 62 or using an excitation light source installed in the light source device. Thus, either the excitation light source or the treatment tool 62 can be omitted from the configuration. In the case of causing excitation of the antibody-drug in photoimmunotherapy, for example, the near-infrared light having the central wavelength of 690 nm is used.

Meanwhile, in the embodiment described above, the explanation is given about an example in which the endoscope system 1 is applied in photoimmunotherapy (PIT). However, the endoscope system 1 is not limited to be applied in photoimmunotherapy, and can be applied in any treatment method in which the treatment is carried out using a photosensitive material, such as in photodynamic therapy (PDT).

Moreover, in the embodiment described above, the endoscope system 1, which treats the body tissue inside a subject as the observation target and which includes the flexible endoscope 2, represents the endoscope system according to the disclosure. Alternatively, it is also possible to use an endoscope system in which either a rigid endoscope is used, or an industrial endoscope is used for observing the characteristic features of materials, or a fiberscope is used, or such a device is used in which a camera head is connected to the eyepiece of an optical endoscope such as an optical viewing tube.

As explained above, the image processing device, the phototherapy system, the image processing method, the computer program product, and the phototherapy method according to the disclosure are suitable in confirming the accumulation of the antibody-drug and confirming the treatment progress while holding down the advancement of the reaction during photoimmunotherapy.

Thus, according to the disclosure, the accumulation of the antibody-drug and the treatment progress can be confirmed while holding down the advancement of the reaction during photoimmunotherapy.

Additional advantages and modifications will readily occur to those skilled in the art. Therefore, the disclosure in its broader aspects is not limited to the specific details and representative embodiments shown and described herein. Accordingly, various modifications may be made without departing from the spirit or scope of the general inventive concept as defined by the appended claims and their equivalents.

What is claimed is:

1. An image processing device comprising a processor comprising hardware, the processor being configured to:
generate a white light image based on a white light which is made of a light having a wavelength band of visible light range;
generate an absorption band light image indicating an absorption degree of a light having a wavelength band of Soret band which is absorbed by a drug accumulated in a target region for treatment; and
generate a display image based on the white light image and the absorption band light image.

2. The image processing device according to claim 1, wherein the processor is further configured to
divide a blue signal by a green signal to standardize a signal value of the blue signal, the blue signal being based on a light having a wavelength band of blue color, the green signal being is based on a light having a wavelength band of green color, and
generate the absorption band light image using the standardized signal value of the blue signal.

3. The image processing device according to claim 1, wherein the processor is further configured to
divide a blue signal by a sum of a signal value of the blue signal, a signal value of a red signal, and a signal value of a green signal to standardize the signal value of the blue signal, the blue signal being based on a light having a wavelength band of blue color, the red signal being based on a light having a wavelength band of red color, the green signal being based on a light having a wavelength band of green color, and
generate the absorption band light image using the standardized signal value of the blue signal.

4. The image processing device according to claim 1, wherein the processor is further configured to
divide a blue signal by a sum of a signal value of a red signal and a signal value of a green signal to standardize a signal value of the blue signal, the blue signal being based on a light having a wavelength band of blue color, the red signal being based on a light having a wavelength band of red color, the green signal being based on a light having a wavelength band of green color, and
generate the absorption band light image using the standardized signal value of the blue signal.

5. The image processing device according to claim 1, wherein the processor is further configured to switch between a white light observation mode in which the white light image is displayed in a display and an absorption band light observation mode in which a display image including an absorption band light image is displayed in the display.

6. The image processing device according to claim 1, wherein
the wavelength band of the Soret band is equal to or smaller than 450 nm, and
a wavelength band for causing excitation of the drug is equal to or greater than 680 nm.

7. A phototherapy system comprising:
a white light emitter configured to emit a white light made of a light having a wavelength band of visible light range;
an absorption band light emitter configured to emit an absorption band light made of a light having a wavelength band of Soret band which is absorbed by a drug accumulated in a target region for treatment;
an image processing device configured to generate an image based on either the white light or the absorption band light; and
a display configured to display the image generated by the image processing device, the image processing device comprising a processor comprising hardware, the processor being configured to:
generate a white light image based on the white light;
generate an absorption band light image indicating an absorption degree of the light having the wavelength band of the Soret band, and
generate a display image based on the white light image and the absorption band light image.

8. An image processing method comprising:
generating a white light image based on a white light which is made of a light having a wavelength band of visible light range;
generating an absorption band light image indicating an absorption degree of a light having a wavelength band of Soret band which is absorbed by a drug accumulated in a target region for treatment; and
generating a display image based on the white light image and the absorption band light image.

9. A non-transitory computer-readable recording medium with an executable program stored thereon, the program causing a computer to execute:
generating a white light image based on a white light which is made of a light having a wavelength band of visible light range;
generating an absorption band light image indicating an absorption degree of a light having a wavelength band of Soret band which is absorbed by a drug accumulated in a target region for treatment; and
generating a display image based on the white light image and the absorption band light image.

10. A phototherapy method comprising:
administering a drug for phototherapy to a target region for treatment;
applying an absorption band light to the target region for treatment to obtain an absorption band light image that indicates an absorption degree of the absorption band light, the absorption band light including a wavelength band of Soret band and having a wavelength band lower than an excitation wavelength of the drug;
generating a display image that includes the absorption band light image;
displaying the display image;
observing the absorption band light image to confirm an amount of accumulation of the drug; and
applying a treatment light onto the target region for treatment to cause the drug that is bound to the target region for treatment to react.
